(19) United States
(12) Patent Application Publication
(10) Pub. No.: US 2017/0361525 A1
(43) Pub. Date: Dec. 21, 2017

(54) Method and Apparatus for Partitioning a Material

(71) Applicants: Benjamin P. Warner, Los Alamos, NM (US); Francesco de Rege Thesauro, Acton, NM (US)

(72) Inventors: Benjamin P. Warner, Los Alamos, NM (US); Francesco de Rege Thesauro, Acton, NM (US)

(21) Appl. No.: 15/619,448

(22) Filed: Jun. 10, 2017

Related U.S. Application Data
(60) Provisional application No. 62/352,269, filed on Jun. 20, 2016.

(51) Int. Cl. B29C 64/112; A61K 9/70; B33Y 10/00; B33Y 70/00; A61K 31/352; A61K 36/185

(52) U.S. Cl. CPC B29C 64/112; A61K 31/352; A61K 36/185; B33Y 10/00; B33Y 70/00; A61K 9/703

(57) ABSTRACT

Methods and apparatus are described for the partitioning of difficult to handle materials such as viscous and sticky materials. The partitioning is accomplished accurately and precisely using an apparatus to extrude the material in portions on or in receptacles disposed on a stage.

### RELATED APPLICATIONS/CLAIM OF PRIORITY

This application claims priority from U.S. provisional application Ser. No. 62/352,269 filed Jun. 20, 2016 which is herein incorporated by reference.

### FIELD OF THE DISCLOSURE

The present disclosure relates generally to partitioning of materials, for example, the portioning of difficult to process viscous materials.

### BACKGROUND OF THE INVENTION

3-D printing is an additive manufacturing process that builds a part in a layer-by-layer fashion to create a three-dimensional object from a digital model. Initially developed in the mid 1980's and used subsequently in highly specialized industries with the expertise and financial means to mitigate the high costs, 3-D printing has recently become a technology that is cheap and accessible to almost anyone. Today's 3D printers include room sized systems but are more typically desktop instruments and can be used for creating and/or prototyping items as disparate as human organ replacements and turbine parts.

Although many materials can be used to make 3D printed parts, many materials are difficult or impossible to use as the print feedstock using the current technologies. There therefore is a need for 3D printers and methods of printing unconventional materials.

### SUMMARY

In general, methods, equipment and systems are described herein for the production of small portions accurately, precisely and repeatedly. For example, a viscous or sticky material can be partitioned into a plurality of non-contacting portions.

In accordance with the invention there is provided a method for partitioning a material. The method includes extruding the material through a CNC controlled nozzle and arraying the material onto a partitionable receptacle forming an array of non-contacting portions. The partitionable receptacle can include a surface for arraying the material upon with a surface energy below about 40 mN/m (e.g., a non-stick receptacle such as wax paper, perforated wax paper, a TEFLON™ treated surface). The partitionable receptacle can also include a container (e.g., a medicinal capsule, a vaporizing pen cartridge, a jar for a cream or for holding wax or oil). Optionally the partitionable receptacle comprises a coupon. The partitionable material can be made of any material, for example, metal, plastic, paper and combinations of these (e.g., laminates). The partitionable receptacle can be a component of a transdermal patch. In some implementations of the method, the material can include a cannabis extract. For example, cannabis extracted from plant material using a solvent such as butane, supercritical carbon dioxide and/or ethanol. The cannabis extract can be selected from the group consisting of cannabigerolic acid (CBGA), cannabichromene acid (CBCA), cannabidiol acid (CBDA), Δ⁹-tetrahydrocannabinolic acid (THCA), cannabinol acid (CBNA), cannabigerol (CBG), cannabichromene (CBC), cannabidiol (CBD), Δ⁹-tetrahydrocannabinol (THC), cannabinol (CBN) and mixtures of these. Optionally the cannabis extract is synthetic. In addition to or optionally, the material can include a terpene. For example, the terpene can be selected from the group consisting of Pinene (e.g., alpha-Pinene, Beta-Pinene), Myrcene, Limonene, Caryophyllene, Linalool, Terpinolene, Camphene, Phellandrene, Humulene, Phellandrene, Phytol, Pulegone, Bergamotene, Farnesene, Delta-3-Carene, Elemene, Fenchol, Aromadendrene, Bisabolene, alpha-Bisabolol, Borneol, Euclyptol, Cineole and mixtures of these. The Terpene can be an extract or made synthetically. Optionally the method includes a medicinal preparation that is consumed by ingestion, by inhalation, by smoking, by sublingual application or by transdermal application (e.g., using a transdermal patch).

In some implementations of the method each non-contacting portion comprises between about 1 mg and 100 g (e.g., between about 1 mg and 1 g, between about 1 mg and 500 mg of material, between about 1 mg and about 200 mg, between about 10 mg and about 100 mg) of material. Optionally, the standard deviation of the average of the masses of the array of non-contacting portions is less than about 10% (e.g., less than about 5%, even less than about 1%). The method can be used as a batch process for the production of non-contacting portions of the material. Optionally, the batches include between 2 and 5000 portions (e.g., between 2 and 1000 portions, between 2 and 500 portions, between 2 and 100 portions). In some implementations of the method, a second CNC controlled nozzle is used for arraying the material. Optionally more CNC nozzles are used, such as three, four, five, six, seven, eight, nine, 10 or more.

In some implementations of the method, the non-contacting portions are produced at an average rate of between about 0.01 and about 10 portions per second wherein the time is measured between the first portion that is extruded and the last portion that is extruded in a batch. Optionally, the material is and has a temperature between about 40 and about 100 degrees Celsius while being extruded (e.g., between about 40 and 80 degrees Celsius). The material can have a viscosity below about 1,000,000 centipoise (e.g., below about 10,000 centipoise) while being extruded. Optionally, the material is made to contact and pass through, on and/or across a flexible applicator after being extruded and prior to being deposited onto the partitionable receptacle. For example, the flexible applicator is selected from the group consisting of a plastic nozzle, a silicone nozzle, a plastic tube and a silicone tube.

In accordance with the invention there is also provided a method of partitioning a material by extruding the material through a CNC controlled nozzle and arraying the material into at least two molds, forming an array of non-contacting portions.

The apparatus described herein can be used for partitioning materials that are liquids and viscous pastes. For example, the materials are useful for partitioning materials that are difficult to partition by hand such as resinous and stick materials that attach to implements such as spatulas. Also, materials that at room temperature are brittle and hard can be difficult to partition by hand and the apparatus described herein can be useful for these materials. The apparatus can also be used to prepare relatively small batch sizes in several locations (e.g., state, province) rather than large continuous processes that produce much larger amounts of materials in a centralized location (e.g., nationally, internationally). Such scale is useful for materials that are highly regulated such as medicinal materials. The scale of the apparatus makes this an economical as well as practical alternative. In addition, the apparatus as described herein has very little dead volume such as long tubes wherein material is wasted and/or requires extensive cleaning. Rather, the material that is to be partitioned is efficiently utilized.

Other features and advantages of the invention will be apparent from the following drawings, detailed description, and from the claims.

### DESCRIPTION OF THE DRAWINGS

The foregoing will be apparent from the following more particular description of example embodiments of the invention, as illustrated in the accompanying drawings. The drawings are not necessarily to scale, emphasis instead being placed upon illustrating embodiments of the present invention.

FIG. 1 is a highly diagrammatic view of an apparatus for partitioning a material.

FIG. 2A shows a top down view of a partitionable receptacle. FIG. 2B shows a side view of the array of portions.

FIG. 3 shows a top down view of a partitionable receptacle with an array of material.

FIGS. 4A and 4B show top down views of arrays of differently shaped material

FIG. 5 is highly diagrammatic view of an apparatus for partitioning a material.

FIGS. 6A and 6B show an extruder.

FIG. 7 is a highly diagrammatic view of an alternative embodiment of an apparatus for partitioning material.

FIG. 8 is a plot of mass to deposited portion for a first test.

FIG. 9 is a plot of mass to deposited portion for a second test.

### DETAILED DESCRIPTION

### Glossary

As used herein, CNC control refers to computer numerical control. For example, where the motions of a machine are controlled by a prepared program containing coded alphanumeric data such as G-code. CNC control can control the motion of a nozzle and stage of an additive manufacturing machine relative to each other (e.g., their relative x, y, z position), other energy outputs (e.g., heating, cooling, electrical power to a laser), weight on a printer bed, optical feeds from digital cameras and speed of extrusion of a feedstock.

As used herein, a linear actuator refers to an actuator that creates motion in a straight linear path.

As used herein; x, y and z are Cartesian coordinate points. X, Y and Z refer to the Cartesian directions. Clearly, other coordinate systems can be used by applying the appropriate transfer function, e.g., to polar coordinates.

As used herein, viscosity is a measure of a liquid's resistance to deformation by shear or tensile stress. Low viscosity liquids have a viscosity of less than about 10,000 centipoise and can be poured (e.g., up to about the consistency of honey at room temperature). Medium viscosity liquids have a viscosity between about 10,000 centipoise and about 1,000,000 centipoise (e.g., pastes including ketchup and peanut butter) and can be extruded with moderate force but cannot be easily poured. High viscosity liquids have viscosity above about 1,000,000 centipoise and are pastes or putties that cannot be poured (e.g., Caulking compounds between about 2 and 5 million cP, window putty more than 100 million cP).

### Embodiments

Using the equipment, methods and systems described herein, and illustrated in the Figures, a difficult to partition material can be partitioned. For example, apparatus and methods are described for partitioning of viscous and sticky materials such as resins and oils.

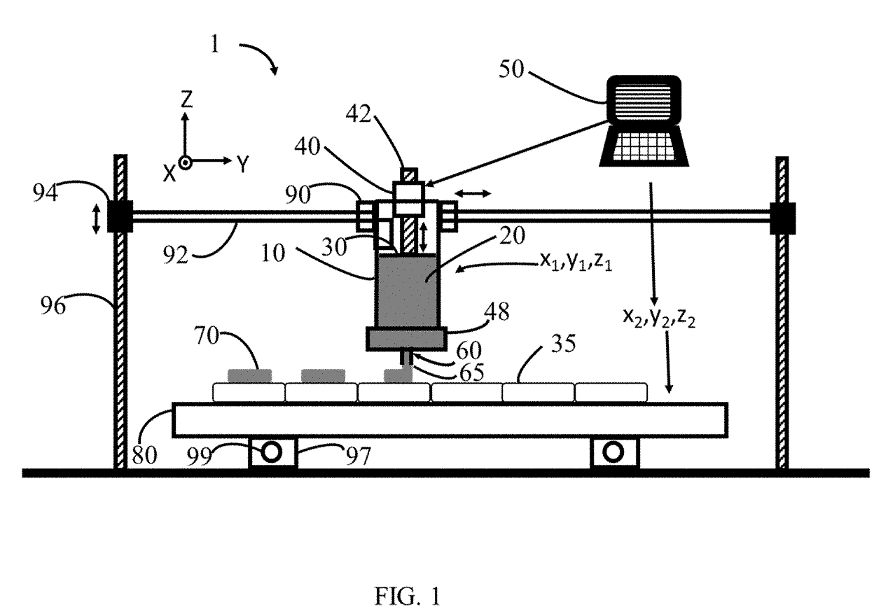

FIG. 1 is a highly diagrammatic view of an apparatus 1 for partitioning a material. The apparatus includes a chamber 10 capable of containing a material 20. The chamber includes at least one movable wall 30. The wall can be made to move by a mechanical device 40 (e.g., a stepper motor) coupled to a screw 42 and nut (e.g., components of a linear actuator) in mechanical communication with the wall. For example, the wall can be made to move up and down in the Z direction as indicated by the double headed arrow next to screw 42. The device 40 can be controlled by a CNC controlling device 50 such as a computer executing an algorithm, and using an appropriate intermediate hardware (e.g., an Arduino motherboard). The chamber 10 also includes at least one opening 60 through which material 20 can be extruded. A nozzle 65 can be attached to the opening. The material 20 can be extruded out of chamber 10 through the opening and deposited as extruded material 70 (e.g., having the same composition as 20). A stage 80 is disposed to receive the extruded material from the nozzle. The stage can be heated (e.g., up to about 120 degrees Celsius) and/or cooled (e.g., down to about −40 degrees Celsius). The stage can include a partitionable receptacle 35 that is placed on the stage and disposed between the stage and nozzle so that material is extruded onto the partitionable receptacle (e.g., a first layer of extruded material is deposited on the receptacle 35 with subsequent layers deposited on previously deposited material). The relative position of the stage 80 and the opening 60/nozzle 65 is controlled by the CNC control, therefore the relative position of the partitionable receptacle 35 and the nozzle is also controlled by the CNC control.

A heater 48 can be included with apparatus 1. For example, the heater can be a heating tape that contacts the chamber, a conducting metal in contact with the chamber and a heating cartridge, a hot air gun directed at the chamber, an IR lamp directed at the chamber, a heating jacket with a heating fluid passing therethrough (e.g., heated water, heated oil, heated air) or another radiative heater in proximity to the chamber. The entire chamber can be heated or a portion of the chamber can be heated. In some embodiments, the heater directs heat near the outlet of the chamber. In some embodiments, the chamber includes an insulated portion distal from the opening and a heat conductive portion proximal to the opening. The heating is monitored, for example by a thermo-couple integrated with the heater or in the chamber, an IR heat detector directed at the chamber or any other useful heat monitoring device (e.g., a thermometer). The heating is controlled by the CNC control.

The CNC control communicates with electrical and mechanical devices that control the relative position of the chamber 10 (e.g., x₁, y₁, z₁) and of the stage 80 (e.g., x₂, y₂, z₂). In addition, the CNC control communicates with the mechanism 40 and thus the extrusion rate out of opening 60 and nozzle 65. The CNC control also controls the temperature of the heater. For example, the CNC controls stepper motors coupled to the stage or chamber through direct screw drives, belts and or pulleys. Gantries, tracks and other methods of smooth movement of the stage and/or chamber relative to each other in X, Y and Z directions can be used. An optional algorithm executes a relative X and Y movement of the opening/nozzle to the stage while extruding material at a specified rate, optionally followed by an incremental movement up in the Z direction, and deposition of another layer by relative movement in the X and Y directions. The algorithm may include pauses in motion, and motions in any direction. Such motions can be used to allow inspection, adjustment, modification, or other actions to be performed on the material being extruded or the apparatus. It is understood that the relative movement of the chamber and stage can be achieved my many different configurations. For example, under CNC control and the electrical and mechanical devices, the stage may move in the X and Y direction and the chamber moves in a Z direction; in another configuration, the stage may move in a Z direction and the chamber moves in an X and Z direction; alternatively, the stage may move in a Y direction while the chamber moves in an X and Z direction; in another option the stage may not move and the chamber may move in X, Y and Z directions. The exact configuration for CNC movement can be selected by the Artisan. In some embodiments such as depicted in FIG. 1, the chamber can be relatively heavy since it supports all the feed material and linear actuator. Therefore, it may require a strong rigid structure made of metal (e.g., aluminum and/or steel).

FIG. 1 shows one possible configuration for movement using a gantry to move the chamber relative to the stage. The gantry has a carriage 90 that is fastened to the chamber. The carriage can move in the Y direction on rail 92. The rail is fastened to nut 94 which is coupled to screw 96 and therefore can move the chamber in the Z direction. Movement of the carriage and screw can be done using stepper motors coupled to the carriage and screw (e.g., direct drive for the screw, through a belt for the carriage). The stage can be moved in the X direction with a second carriage 97 and rail 99. Other configurations include a stage that does not move and a gantry with 3 orthogonal rails to move the chamber in X, Y and Z directions are conceived.

As previously described, the movable wall 30 can be moved by means of a linear actuator that is in mechanical communication and/or contact with the wall. For example, as shown in FIG. 1, the actuator is in contact on one side of the wall while the other side is in contract with the material. Although a screw and nut is depicted in FIG. 1, any suitable linear actuator can be utilized. Preferably the linear actuator can be selected from the group consisting of a screw and nut, a pneumatic or hydraulic piston, a solenoid, a wheel and axle or a cam. For example, by rotation of an actuator nut relative to a screw, the screw can move in and out of the threaded hole in a linear fashion (e.g., or the nut moves up or down the shaft). In an alternative, a wheel and axle can be coupled to a belt that is also connected to a rigid shaft and can move the shaft in a linear fashion. Also, a cam can be used to provide thrust at the base of a shaft.

Mechanisms other than a linear actuator are recognized for moving the walls of the chamber. For example, the Tube-Wringer® (Gill Mechanical Co., Oregon) acts by squeezing two walls of a flexible tube (e.g., configured as a toothpaste or caulking tube) between rollers. Such rollers could be modified to be driven by a motor and CNC controlled. Alternatively, more than one linear actuator could be used, for example, pushing on two walls of the chamber, such as opposing sides of a flexible tube.

The equipment, methods and apparatus herein preferably have very little dead volume. That is, at least 90 vol. % (e.g., at least 95 vol. %, at least 99 vol. %) of the contents (e.g., 20 in FIG. 1) in the chambers (e.g., 10) can be extruded out through the nozzle.

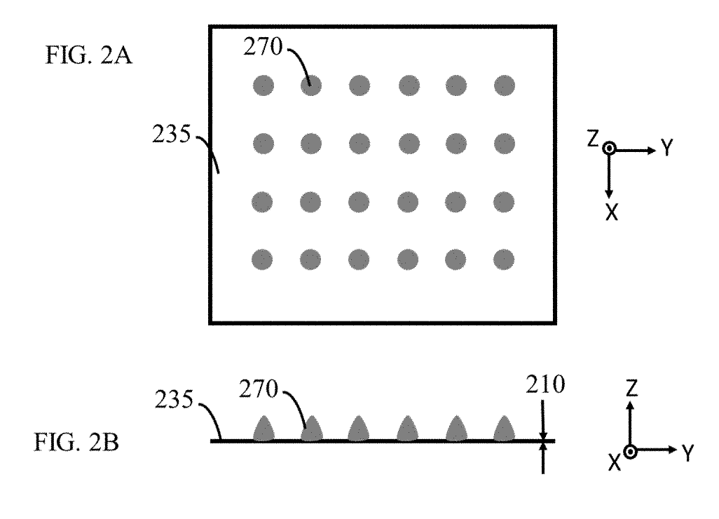

FIG. 2A shows a top down view of a partitionable receptacle 235 with material 270 deposited as an array of portions. FIG. 2B shows a side view of the array of portions. The partitionable receptacle can be a thin sheet such as a sheet of metal foil (e.g., aluminum foil), plastic (e.g., cellophane), paper (e.g., wax paper) or a foodstuff (e.g., a crepe.). For example, the sheet can of a thickness 210 between about 0.1 mm and about 5 mm. The sheet is preferably easy to partition by hand, for example, by tearing or cutting with a blade (e.g., scissors, Guillotine cutters, rolling cutter).

In some optional embodiments, the receptacle is a non-stick receptacle such that it has at least one surface having a low energy disposed for receiving the depositing material. For example, the low energy surface can have a surface energy below about 40 mN/m. Preferably the low energy surface has a surface energy between about 20 mN/m and about 40 mN/m. Most preferably the non-stick receptacle has a surface energy between about 25 mN/m and 28 mN/m. Without being bound to a specific mechanism, it is believed that having a too high surface energy will make the associating of the deposited material to the receptacle strong, and therefore separation of the two can be rendered difficult in further processing. It is also recognized that having a surface energy that is too low can make the association of the depositing material and the receptacle too weak so that poor deposition occurs.

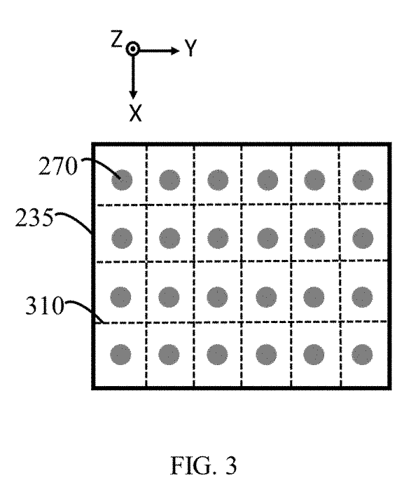

FIG. 3 shows a top down view of a partitionable receptacle with an array of portions. The partitionable surface is a sheet that has been perforated wherein the perforations are shown as dashed lines 310. The perforations can facilitate the portioning of the receptacle as well as help organize the array of deposited material. Other methods are envisioned that can serve this purpose, such as lines drawn on the receptacle (e.g., etched, painted or drawn) and/or methods of weakening the receptacle (e.g., to facilitate it's partitioning) such as etching and scouring. Optionally, the partitional receptacles are completely scored through or separate parts that are placed next to each other such as containers, individual sheets or components (e.g., components of transdermal patches).

The material can be deposited as a regular array of material as shown above. For example, FIGS. 2A and 3 show 4 rows by 6 columns of portions, or 24 partitions of the material 270. The 24 partitions can be considered a batch and after a batch of material has been extruded, the partitionable receptacle is removed and a new partitionable receptacle can be supplied to the machine so that additional batches can be made. Batches can include one or more partitions and depend in part on the size of each partition as well as the size of the partitionable receptacle. The partitioning is precise and accurate and the amount of material in each portion is determined by the operator through use of the CNC control. For example, materials can be partitioned into portions of greater than about 1 mg (e.g., greater than 10 mg, greater than 50 mg) and as large as the volume of the apparatus allows (e.g., 1 Kg). Accuracies of greater than +/−10 mg (e.g., greater than +/−5 mg, greater than +/−1 mg) are readily achieved. In some embodiments, the individual portions weigh between about 10 mg and about 100 g (e.g., between about 1 mg and about 50 g, between about 1 mg and about 10 g, between about 1 mg and 1 g, between about 1 mg and 500 mg of material, between about 1 mg and about 200 mg, between about 10 and about 100 mg). Also, in some embodiments the batches include between 2 and 5000 portions (e.g., between 2 and 1000 portions, between 2-500 portions, between 2-100 portions).

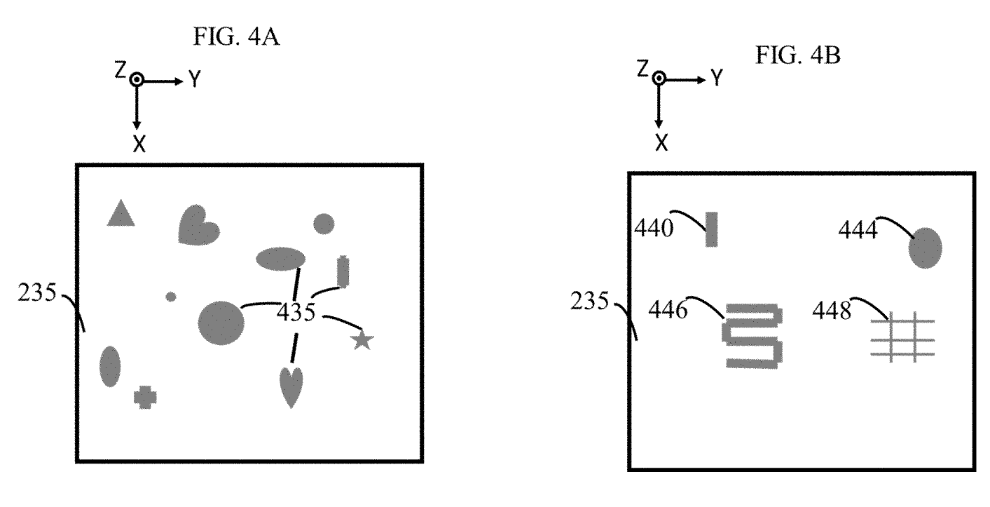

Although the embodiments show a regular array of deposited material, the material can be deposited in an irregular array as determined by the operator and/or designer of the run and implemented by the CNC control of the apparatus. In addition, the embodiments show deposition of material with similar shapes. It is envisioned that the material can be deposited in arrays of different shapes. For example, FIG. 4A shows a top down view of an irregular array of differently shaped material 435 deposited on a partitionable receptacle 235.

In some embodiment only a single layer of material is extruded per non-contacting portion. The portions can also have a high width to height aspect ratio. For example, as shown in FIG. 4B (top down view) material can be deposited as long flat strips 440 such as sub-lingual strips or gum strips, medallions 444, serpentine shapes 446, and even lattices 448. The portions, such as those shown in FIG. 4B, can have a width to height ratio of greater than about 2 and more preferably greater than about 5 (e.g., greater than about 10); wherein the width is measured as the diameter of a circle drawn parallel to the XY plane that contains the non-contacting portion such as 440, 444, 446 or 448, and the height is the maximum distance perpendicular to the circle containing the non-contacting portion.

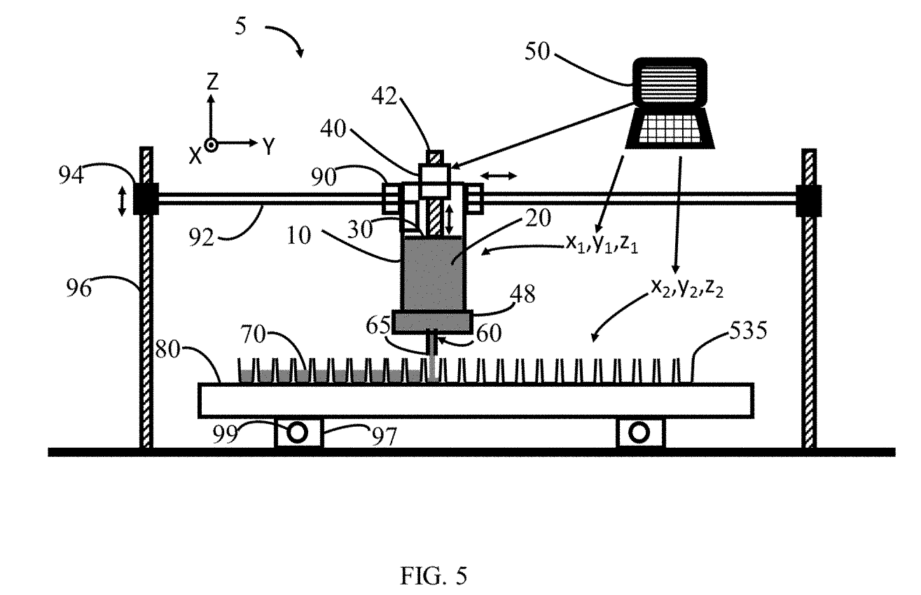

FIG. 5 is highly diagrammatic view of an apparatus 5 for partitioning a material. The partitionable receptacle is configured as a plurality of containers 535. The stage 80 can provide a flat surface for placement of the containers, or the stage can be configured with fixtures for placing and/or holding the containers in specific locations. For example, the fixture can include an indentation that the container fits in. Guiding lines can also be scribed, etched or drawn on the stage to indicate where containers should be placed. Guiding lines can also be scribed, etched or drawn on a jig or tray placed on the stage to indicate where containers should be placed. Containers that can be used in the apparatus include cartridges (e.g., vaporizing pen cartridges) and capsules (e.g., drug capsules).

Other embodiments include the partitionable receptacle configured as a portion of a transdermal patch. For example, the partitionable receptacle can be a release liner, the backing layer or a rate controlling membrane. Alternative embodiments include the partitionable surface configured as metal (e.g., titanium) and ceramic (e.g., glass, quartz) coupons or containers where upon the material is deposited in non-contacting portions.

It is understood by the artisan that a non-contacting portion might have a small amount of contact. For example, some materials can form thin strands that bridge two or more of the non-contacting portions. This can occur with very viscous and sticky materials such as resins and gums. In these cases, the amount of material in such contacting strings are less than about 1 wt. % of the material in the non-contacting portion.

Although control of the portion amounts can be controlled by flow rates, a feedback mechanism including weighting the portions or optically observing the portions while they are extruded is envisioned. For example, a single or an array of piezoelectric devices placed under the partitionable receptacle and on the stage, that detect the weight of material as it is extruded. The signal from the piezoelectric devices can be fed back to the CNC control which modulates an extruding mechanism such as 40. Similarly, optical detection of the extruded amount can be implemented by a digital camera and the images compared to expected profiles. For example, if the partitionable receptacle is configured as a container, the level of filling can be detected optically. Alternatively, the weighing and/or optical device can be passive and record the amount of material deposited as the operation proceeds and thus determine if the deposition process is within acceptable parameters. Portions that are not within acceptable limits can be discarded or recycled.

In some embodiments, the chamber and movable wall are configured as a syringe, with the barrel of the syringe defining the chamber and the movable wall being the surface of the plunger placed inside the barrel. In optional embodiments, the syringe is partially or completely disposable. For example, the syringe can include a lining, tube or a cartridge that is disposable.

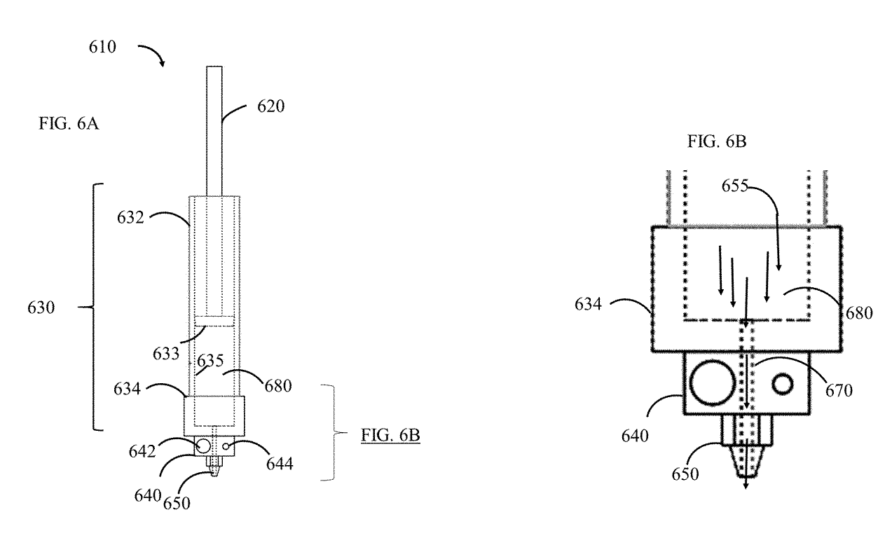

An embodiment of the chamber configured as a heated syringe 610 is shown in FIG. 6A as a cross cut view. The plunger 620 fits in the barrel 630. The plunger surface 633 and internal surfaces of barrel 635 defines the chamber 680 that can contain a material to be extruded. The barrel preferably includes a portion that is made of an insulating material 632 (e.g., a plastic, silicon glass, ceramic) and a heat conductive material 634 (e.g., a metal such as aluminum or stainless steel); alternatively, the barrel may be entirely made of a conducting material or an insulating material. A heating block 640 is attached to the heat conductive material. The heating block includes a cartridge or a resistive heater 642 and a thermocouple 644. Wires to the heating cartridge and thermocouple are not shown. The heating block is preferably made of a heat conducting material. A nozzle 650 is attached to the heating block. For example, the nozzle and heating block can include complementary threading so that the nozzle can be screwed into the heating block. The nozzle can be made of a heat conducting material or an insulator. FIG. 6B shows a magnified view of the extruding end of the heated syringe. A channel 670 passes through 634, 640 and 650. Therefore, the contents of the chamber 680 are in fluid communication through the heating block and nozzle through channel 670 and material can be made to extrude from the chamber, through the heating block and through the nozzle as indicated by the arrows 655 in FIG. 6B.

Is some embodiments, the chamber portion 632 (FIG. 6A) is disposable. The chamber portion 632 can be removably connected to chamber portion 634, for example such that 632 fits into 634 and the two are complementarily threaded and/or held together by friction and/or fasteners. Therefore, once material has been extruded from the chamber, 632 can be removed (e.g., by unscrewing from 643) and a new chamber portion 632 that is charged (e.g., full of the desired extruding material) can be attached to the 634 and extrusion can then be resumed.

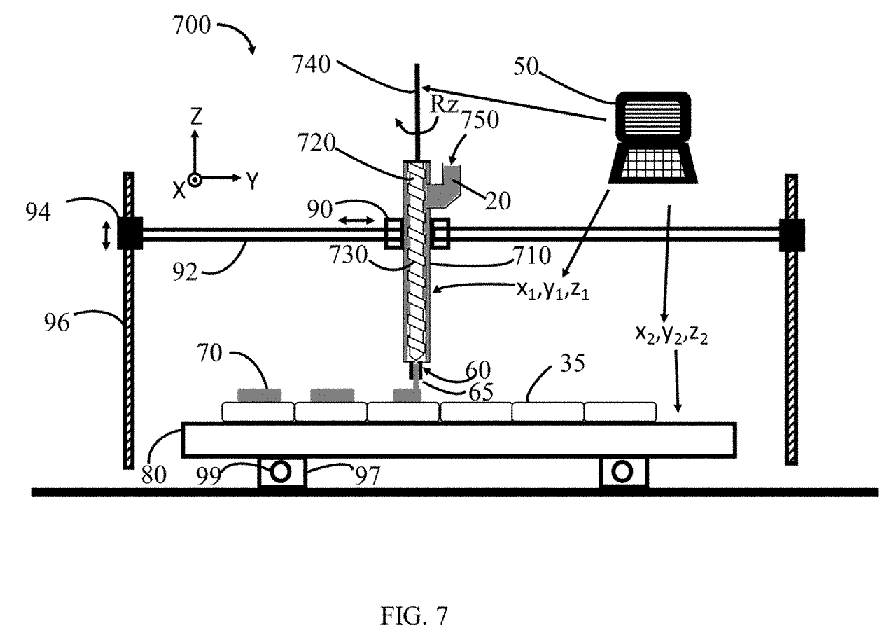

FIG. 7 exemplifies another embodiment of an apparatus 700 for portioning material wherein the movable wall can be the screw of an extruder. For example, the screw extruder (shown as a cross cut view) has a chamber 710, containing the screw 720. The flights of the screw make a moving wall 730 in the chamber. A mechanism for moving the screw (e.g., the wall) can be a rotatable shaft 740 e.g., rotating in the direction indicated by curved arrow Rz. A drive motor can be coupled to the shaft to have it rotate around its axis (the drive motor is not shown). The rotation speed can be controlled by the CNC controller 50. The extruder can be continuously fed through an extruder ingress 750, for example where the ingress is coupled to a feed-hopper. Other features are the similar as indicated in FIG. 1; Material 20 in the chamber 710, extruded material 70, opening 60, nozzle 65, stage 80. In the embodiment shown, gantry with carriage 90, rail 92, nut 94, screw 96, rail 99 for the stage and carriage 97 for the stage is shown as previously described. Other similar embodiments include using a progressive cavity pump in place of the screw extruder.

In some embodiments two or more chambers are used and each chamber feeds the material to be extruded through an opening in each chamber, to the nozzle inlets. Therefore, between the outlet of the chambers and the nozzle inlets the two materials combined prior to being extruded through the nozzle. The location or region where the combination occurs is an in-line mixer. For example, with two chambers, the mixer can be in a “Y”-shaped configuration wherein the mixing chamber has two inlets connected to the outlets of the chambers and one outlet connected to the nozzle inlet. The size of the inlets to the chamber can be each of different sizes, for example to control the amount of material from each chamber allowed into the mixing chamber. The chamber can be an elongated tube, elliptical, rectangular, conical or any other suitable shape. Mechanical mixing such as rotating propellers, paddles, rotor stators and/or turbines can be used to improve the mixing. Mechanical stationary means such as a static mixer can also be used. Preferably, a static in-line mixer is used. In other embodiments two or more chambers with each having a corresponding outlet and nozzle can be utilized, for example, such as to produce non-contacting portions faster due to the possible parallel processing of material.

The materials that can be partitioned using the apparatus described herein include liquids with low, medium and high viscosity. Preferably the materials have medium to low viscosities at room temperature. If the materials have a medium viscosity at room temperature, it is preferably the materials have a low viscosity at an elevated temperature (e.g., between about 40 and about 100 degrees Celsius, between about 40 and 80 degrees Celsius).

In some optional embodiments, the receptacle is not partitionable but the material can be easily removed from or detached form the receptacle. For example, the receptacle can be one or more molds. For example, the mold can include an array of 2 or more shapes, each of which can be filled with material to make a non-contacting portion (e.g., an array of 2, 3 or 4 shapes makes 2, 3 or 4 non-contacting portions respectively). Alternatively, an array of molds can be placed on the stage. The mold can also be shaped from any suitable material such as plastics, silicones and cellulosic materials. The mold can even be stamped into an appropriate powdered material such as corn starch. A releasing agent can be applied to the mold such as cornstarch and/or the surface disposed for contacting the material has a low surface energy such below about 40 mN/m (e.g., below about 30 mN/m).

The embodiments include using materials that include cannabis extracts. There has been a growing interest and public acceptance of the use of cannabis for medicinal and recreational use. The plant material has been used for their therapeutic effects in treating the symptoms of cancer, aids, multiple sclerosis, pain, glaucoma, epilepsy and other conditions. In the plant, some of the active components include cannabigerolic acid (CBGA), cannabichromene acid (CBCA), cannabidiol acid (CBDA), Δ⁹-tetrahydrocannabinolic acid (THCA) and cannabinol acid (CBNA). These can be used in creams, eye drops, therapeutic patches, edible pills and by heating the material and inhaling the smoke such as through a cannabis cigarette or pipe. Heating cannabinoids decarboxylates the components described above producing cannabigerol (CBG), cannabichromene (CBC), cannabidiol (CBD), Δ⁹-tetrahydrocannabinol (THC) and cannabinol (CBN) and volatilizes the components. In addition to the above, cannabis extracts also include many other ingredients such as terpenes. For example; Pinene (e.g., alpha-Pinene, Beta-Pinene), Myrcene, Limonene, Caryophyllene, Linalool, Terpinolene, Camphene, Phellandrene, Humulene, Phellandrene, Phytol, Pulegone, Bergamotene, Farnesene, Delta-3-Carene, Elemene, Fenchol, Aromadendrene, Bisabolene, alpha-Bisabolol, Borneol, Euclyptol, Cineole and mixtures of these. In addition to smell and taste, these auxiliary components purportedly can provide synergistic medicinal properties. Excessive and/or prolonged heating of these terpenes can volatilize them removing them from the extract which can be detrimental to the efficacy of the extract.

The above extracts can be combined with other ingredients such as sugar, starch, oils, fats (e.g., vegetable fats) and jelly prior to portioning. Preferably the materials are not heated above about 120 degrees Celsius while being extruded. For example, the material can be extruded at temperatures between about room temperature and 100 degrees Celsius (e.g., between about 40 and about 100 degrees Celsius, between about 40 and 80 degrees Celsius). In addition, preferably the material is not heated for prolonged periods of time, such as for less than about 30 min (e.g., less than about 20 minutes, less than about 10 minutes)

### Exemplification

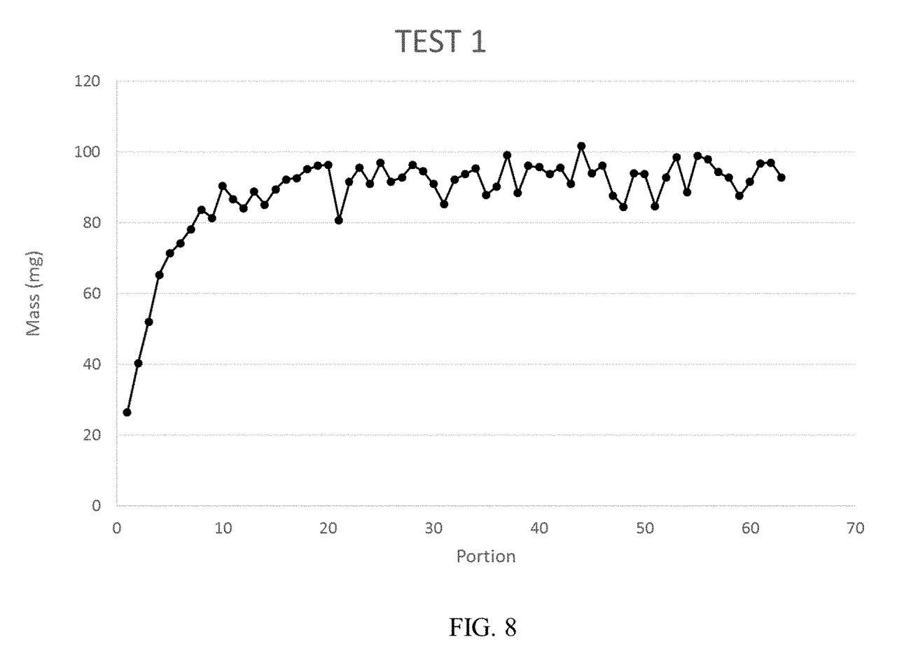

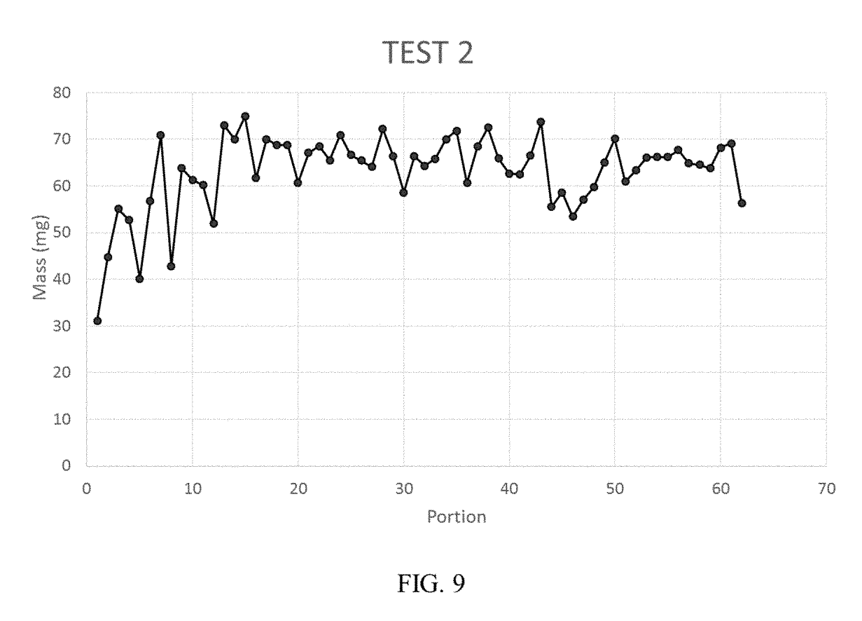

An extruder such as described by FIG. 6 and having a chamber volume of about 60 mL was attached to a CNC mill having a bed size of 30×50 cm. The extruder was attached to the Z axis of the CNC mill in place of the drilling tool. An Arduino 2560 board was electrically connected to the stepper motors of the Z, X, Y and Extruder. A Marlin open source code was used to flash the board and Simplify 3D slicer program was used to prepare the g-code for a 9×7 array of 63 cylinders each having a diameter of 5 mm and 1 mm height. The extruder was loaded with caramel (Kraft™ Caramels). The heater was set to 100 degree Celsius and caramel was extruded onto wax paper. Two tests were conducted using different extrusion rates. A plot of the weight to portion is shown for each test as FIG. 8 and FIG. 9. After discarding outliers (first 9 portions deposited) the first test gave an average weight per portion of 92 mg with a standard deviation of 4 mg and a range of 21 mg; while the second test gave and average portion of 65 mg with a standard deviation of 5 mg and a range of 23 mg.

Other than in the examples herein, or unless otherwise expressly specified, all the numerical ranges, amounts, values and percentages, such as those for amounts of materials, elemental contents, times and temperatures of reaction, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the standard deviation found in its underlying respective testing measurements. Furthermore, when numerical ranges are set forth herein, these ranges are inclusive of the recited range end points (e.g., end points may be used). When percentages by weight are used herein, the numerical values reported are relative to the total weight.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The terms “one,” “a,” or “an” as used herein are intended to include “at least one” or “one or more,” unless otherwise indicated.

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

While this invention has been particularly shown and described with references to embodiments thereof, it will be understood by those skilled in the art that various changes in form and details may be made therein without departing from the scope of the invention encompassed by the appended claims.

What is claimed is:

1. A method of partitioning a material, the method comprising;
extruding said material through a CNC controlled nozzle and arraying said material onto a partitionable receptacle forming an array of non-contacting portions.

2. The method of claim 1, wherein the partitionable receptacle has a surface for arraying said material upon with a surface energy below about 40 mN/m.

3. The method of claim 1, wherein the partitionable receptacle comprises a container.

4. The method of claim 3, wherein the partitionable receptacle is a medicinal capsule, a vaporizing pen cartridge, a container for a cream, a container for holding wax or a container for holding oil.

5. The method of any one of claim 1, wherein the partitionable receptacle comprises a coupon.

6. The method of claim 1, wherein the partitional receptacle comprises metal, plastic, paper or a combination of these.

7. The method of claim 1, wherein the partitionable receptacle is a component of a transdermal patch.

8. The method of claim 1, wherein the material comprises a cannabis extract.

9. The method of claim 8, wherein the cannabis extract is selected from the group consisting of cannabigerolic acid (CBGA), cannabichromene acid (CBCA), cannabidiol acid (CBDA), Δ⁹-tetrahydrocannabinolic acid (THCA), cannabinol acid (CBNA). cannabigerol (CBG), cannabichromene (CBC), cannabidiol (CBD), Δ⁹-tetrahydrocannabinol (THC), cannabinol (CBN) and combinations thereof.

10. The method of claim 1, wherein the material comprises a terpene.

11. The method of claim 1, wherein the terpene is selected from the group consisting of Pinene, alpha-Pinene, Beta-Pinene, Myrcene, Limonene, Caryophyllene, Linalool, Terpinolene, Camphene, Phellandrene, Humulene, Phellandrene, Phytol, Pulegone, Bergamotene, Farnesene, Delta-3-Carene, Elemene, Fenchol, Aromadendrene, Bisabolene, alpha-Bisabolol, Borneol, Euclyptol, Cineole and combinations thereof.

12. The method of claim 1, wherein each portion comprises between about 1 mg and 100 g of material.

13. The method of claim 1, wherein the standard deviation of the average of the masses of the array of non-contacting portions is less than about 10%.

14. The method of claim 1, wherein the method is a batch process.

15. The method of claim 1, wherein the portions are produced at a rate of between about 0.01 and about 10 portions per second.

16. The method of claim 1, wherein the material is heated and has a temperature between about 40 and about 100 degrees Celsius while being extruded.

17. The method of claim 1, wherein the material has a viscosity below about 1,000,000 centipoise while being extruded.

18. The method of claim 1, wherein the portions have an average width to average height ratio of greater than about two.

19. The method of claim 1, wherein the material is made to contact and pass through a flexible applicator after being extruded and prior to being deposited onto the partitionable receptacle.

20. A method of partitioning a material, the method comprising;
extruding said material through a CNC controlled nozzle and arraying said material into at least two molds forming an array of non-contacting portions.
